(19) United States
(12) Patent Application Publication
(10) Pub. No.: US 2010/0323978 A1
(43) Pub. Date: Dec. 23, 2010

(54) Non-aqueous oil delivery system for ophthalmic drugs

(76) Inventor: Calvin Hanna, Little Rock, AR (US)

Correspondence Address: CALVIN HANNA, 74 GLENMERE, LITTLE ROCK, AR, 72204, US

(21) Appl. No.: 12/456,709

(22) Filed: Jun. 22, 2009

(51) Int. Cl. A61K 31/7048; A61K 31/137; A61K 31/4178; A61K 31/215; A61K 31/135; A61K 31/497; A61K 31/164; A61K 31/24; A61K 31/4164; A61K 31/44; A61P 27/02

(52) U.S. Cl. 514/29; 514/36; 514/653; 514/397; 514/530; 514/649; 514/253.04; 514/608; 514/535; 514/385; 514/357

(57) ABSTRACT

The present invention relates to a delivery system for ophthalmic drugs, and more particularly, to a non aqueous oil delivery system. Low concentrations of ophthalmic drugs suspended in an oil vehicle delivery system are as therapeutically effective in man and animals as the corresponding higher concentrations of ophthalmic drugs that are commercially used in aqueous solutions. Eye drops that utilize this nonaqueous oil delivery system, when used in man, are comfortable to use and produce little ocular irritation, have a longer shelf-life, low systemic toxic potential, and only short term blurring of vision. Using this nonaqueous oil delivery system, a single drop of ophthalmic drug with a concentration that is 10 times less than the same drug used in commercially available aqueous eye drops is as effective as the commercially available aqueous ophthalmic eye drops that require many applications to be effective. In addition, utilizing the nonaqueous oil delivery system as eye drops produces no ocular sensation of burning, stinging or excessive tearing that is commonly associated with the commercially available aqueous eye drops. This very dilute ophthalmic drug preparation has a greatly reduced systemic toxicity potential as compared to the commercially used aqueous ophthalmic drops. The vehicle includes castor oil, corn oil, glycerol, mineral oil USP, vegetable oil, white petrolatum USP, and mixtures, there of. The ophthalmic class of drugs includes antimicrobials, miotics, mydriatics, mydriatic-cycloplegics, mydriatic-cycloplegic reversal agents and topical anesthetics.

### BACKGROUND OF THE INVENTION

Described herein relates to the field of oil vehicles used to suspend ophthalmic drugs. More particularly, it relates to the field of vehicles used to suspend ophthalmic eye drops. It is especially suited for suspending classes of drugs including antimicrobials, miotics, mydriatics, mydriatic-cycloplegics and mydriatic-cycloplegic reversal agents, topical anesthetics for topical use on man and animal eyes. The vehicles are directed for the most part toward the use of very dilute ophthalmic drugs as eye drops that are potent, comfortable to use and has little systemic toxic potential. There are no non-aqueous oil suspended ophthalmic drugs on the commercial market.

The term % (v/v) or a similar phrase means the percent volume per volume. Percentage concentration of a liquid substance usually expressed in milli Liter (mL) of the solid dissolved in 100 mL liquid.

The term % (w/v) or similar phrase mean the percent weight per volume. Percentage concentration of a solid substance usually expressed in grams (g) per 100 mL of liquid.

The term ‘mm’ means the size in millimeters.

The term ‘in’ means the size or distance in inches or ‘cm’ means the size or distance in centimeters.

Cycloplegia is paralysis of the ciliary muscle; that is, paralysis of accommodation. A cycloplegic is a drug that causes cycloplegia.

Mydriasis is dilation of the pupil. A mydriatic is a drug that causes mydriasis.

Miosis is constriction of the pupil. Miotic is a drug that causes miosis.

The term medicament means a formulation that is useful in the practice of eye care, a diagnostic agent, or that promotes recovery from an ailment.

The term comfort or bland or any similar word means involving essentially no adverse reaction after topical application to the eye.

Toxic refers to an untoward effect after the use of eye drops.

### BACKGROUND REFERENCES

There are at least three OTC ophthalmic products on the market that contain castor oil as an emollient. These eye drops used to ameliorate dry eye symptoms contain some castor oil used as emollients. These products contain as active ingredients various viscosity agents; such as, methylcellulose. Schoenwald, et al. U.S. Pat. No. 4,623,664 suspended phenylephrine HCl and phenylephrine free base in sesame seed oil in concentrations of 0.5 Molar, 0.15 Molar and 0.45 Molar. These suspensions were used as eye drops as 10 micro Liters to eight rabbits. In the compounding section of this Patent phenylephrine free base was suspended after four or more excipients were dissolved by warming the sesame seed oil. Next, a wetting agent (polyethylene glycol) was mixed with the phenylephrine free base and stirred to form a suspension. The excipients were several preservatives, stabilizing agent and antioxidant (0.66% w/v). In the human the sesame seed oil (pure) in amounts of 7 mg produces intense burning and stinging sensation as well as tearing. The phenylephrine free base at 0.15 Molar produced mydriasis in the rabbit.

The 0.5 Molar phenylephrine HCl salt was slightly effective as a mydriatic while the 0.15 Molar phenylephrine HCl was ineffective. Aqueous solutions of phenylephrine HCl are commercially used at 2.5% and 10% w/v. The 0.5 Molar phenylephrine HCl salt is around 11% w/v which is several times less effective than the commercially used 2.5% aqueous solutions of phenylephrine HCl. These results teach that phenylephrine HCl suspended in oil leads to less effective composition. Schoenwald et al. U.S. Pat. No. 4,705,798 further studied phenylephrine free base in sesame seed oil as well as phenylephrine prodrug (oxazolidine of phenylephrine). These two Patents claim the use of a mineral oil' as well as 14 edible oils including olive oil as oil vehicles. However, pure olive oil and sesame seed oil at a volume of 7 mg produced intense burning and stinging as eye drops in man and animals. The results of Schoenwald et al. teach away from the use of phenylephrine HCl suspended in sesame seed oil.

In a later U.S. Pat. No. 4,879,304 Schoenwald employed polydimethylsiloxane to dissolve phenylephrine prodrug (oxazolidine of phenylephrine). Schoenwald noted that ‘In order for a pharmaceutical vehicle to be used as an ophthalmic vehicle it is necessary that it be non-irritating to the eye and non-toxic. In order for it to be most effective it must not blur vision. Never the less known oil vehicles blur the eye. Known vehicles are vegetable oils . . . or mineral oil’. Sawaya amplifies this conclusion in U.S. Pat. No. 5,888,493 by noting that oil cannot be used as an ophthalmic vehicle, “None of these previously patented oil vehicles by Schoenwald, et al. satisfy the three above criteria since they blur vision” and “Cannot use an oil vehicle . . . because oil fluids are rejected by the eye.”

The above studies by Schoenwald and statements by Schoenwald and Sawaya teaches away from the use of oil as a vehicle when used as an eye drop to deliver topical drugs as a suspension in eye drops. The current general consensus is that a non-aqueous oil suspension of drugs in eye drops cannot be used.

Knepper, U.S. Pat. No. 4,812,448 repeatedly injected rabbit eyes with dexamethasone until the intra-ocular pressure increased. An aqueous suspension of alpha-hydroxy-testosterone was injected into the eyes for three days. The intra-ocular pressure decreased. Knepper suggested suspending the injected drug in mineral oil, petrolatum-lanolin, or use a plastic insert.

Kaufman, U.S. Pat. No. 4,865,846 incorporated ophthalmic drug from a solution into plastic particles. These particles were suspended in silicone oil, mineral oil, white oil, corn oil, peanut oil and the like. The bioerrodible plastic particles were dispersed in ointment and water was retained in the plastic particles.

MacKeen, U.S. Pat. No. 5,366,739 used ointment suspended drugs applied to the eye lids of patients in a manner previously published by Wallace, et al. and obtained the same results.

Chandrasakara, et al. U.S. Pat. No. 5,188,886 employed a topical ophthalmic suspension such as phenylephrine in an aqueous gel.

Deurer, et al. U.S. Pat. No. 5,869,086 injected pilocarpine preparations into the skin of man (transdermal) to treat glaucoma. A reservoir, a plastic containing 1-30 wt % pilocarpine was used. A suggestion was made to use a suitable solution or suspension for the pilocarpine injection; for example, water, silicone fluid, or mineral oil for the transdermal injection.

Ali, et al. U.S. Pat. No. 5,461,081 claimed a universal ophthalmic vehicle using an aqueous gel that expands on reaching the eye. A suspension was made of water insoluble drugs in an aqueous polymer composition.

Jemenez-Bayardo, et al. U.S. Pat. No. 6,071,858 used ophthalmic drugs dissolved to give a solution.

Morishima, U.S. Pat. No. 6,288,049B1 suspended the nearly water insoluble fluorometholone in cellulostic or modified polymer of castor oil which is a nonionic surfactant in an aqueous gel.

Aukunuru, et al. WO/2004/009056 invented a staurzsponne derivative for drugs, an agent for dispersing and/or dissolving said drug in an ointment base made from polyethylene glycol or polyethoxylated castor oil.

Sugumoto, et al WO/2005/0923315 claimed an ophthalmic preparation for drugs hardly soluble in water by using a combination of castor oil, cellulose, benzyl alcohol and water.

Ogidigben, et al. WO/2008/027341 employed an oil, a surfactant, tonicity agent to disperse insoluble drugs as aqueous ophthalmic formulations.

### SUMMARY OF INVENTION

Oil suspended eye drops of ophthalmic drugs were found to be comfortable to use with increased (at least ten times) potency, with stability, and can produce clinically useful response when used as a single eye drop. The potential systemic toxicity is many times less when compared to commercially available eye drop medicaments.

### DETAILED DESCRIPTION OF THE INVENTION

Studies were undertaken to determine the value of suspending ophthalmic drugs in oil when used as eye drops. The study was in two parts. Initially four Caucasian volunteers, who self administered eye drops, were used. Later additional volunteers were employed. In each case the volunteers did not know which drug would be used. However, the studies began with a much lower concentration of drug than used in the corresponding commercial eye drops.

The first undertaking involved the use of a commercial aqueous preparation and to compare this with the corresponding eye drop wherein the drug (salt) was suspended in an oil vehicle. The vehicles were first tested in minute volumes such as 7 mg. For example, 7 mg of olive oil or sesame seed oil produced a stinging and burning sensation and was not used further. The drugs were tested to produce mydriasis and cycloplegia, miosis, topical anesthesia and others; such as, antimicrobials.

The second project was to study the miotic and cycloplegic blocking effectiveness of dapiprazole HCl or pilocarpine HCl alone and after the use of the mydriatic and cycloplegic agents such as tropicamide and Paremyd. This study is called reversal.

### Results

In the following results section the commercial medicaments are listed followed by the corn oil and vegetable oil vehicles that are used: Cyclopentolate HCl, USP, ophthalmic solution 1% Bausch and Lomb®, boric acid, potassium chloride, sodium edetate, benzalkonium chloride 0.01%, pH 2.0-5.5. Erythromycin, USP ophthalmic ointment 0.5% E Fougera, mineral oil, white petrolatum, methyl and propylparabens. Gentamycin sulfate, USP, 0.5%, E Fougera, white petrolatum, methyl and propylparabens. Phenylephrine HCl, USP ophthalmic solution 2.5% Bausch and Lomb®, boric acid, sodium phosphates, benzalkonium chloride, water. Paremyd®, Akorn®, hydroxyamphetamine HBr, USP, 1%, tropicamide, USP, 0.25%, ophthalmic solution, sodium chloride, sodium edetate, benzalkonium chloride 0.05%, water. Pilocarpine HCl, USP, 1.0% ophthalmic solution, Bausch and Lomb®. Rev-eyes, dapiprazole HBr, ophthalmic solution, Bausch and Lomb® vial one contains 5 mL of water and vial two contains dapiprazole HCl, 0.5%, sodium phosphate sodium edetate, hypomellose, mannitol, sodium chloride, benzalkonium chloride. Sulfacetamide sodium, USP, 10% ophthalmic solution, Bausch and Lomb®, methylcellulose, sodium phosphate, sodium thiosulfate, methyl and propylparabens, pH 6.8-8.8. Tetracaine HCl USP, 0.5% ophthalmic solution, Bausch and Lomb®. Tropicamide, USP, 0.5% and 1.0% ophthalmic solution, Bausch and Lomb®, boric acid, sodium edetate, benzalkonium chloride, pH 4.0-5.8. Visine® advanced relief, USP, Pfizer®, dextran 70, propylene glycol 400, povidone, tetrahydrozoline HCl, methylcellulose, sodium chloride, sodium edetate, and benzalkonium chloride. Mineral oil, USP, White petrolatum, USP. Corn oil, Kroger Co. Vegetable oil, Wesson Co. glycerol, USP. Castor oil, USP, Pure sesame seed oil, mixing pot foods, Austin, Minn., Olive oil, Bella, Tampa, Fla.

The results below have abbreviations as follows: hydrochloride, hydrobromide, sulfate, and sodium salts are indicated by HCl, HBr, SO4, and Na, respectively. Pupil diameter in millimeters is Pupil div (mm), accommodation in centimeters is Accomm (cm), corneal reflex is the blinking response or no reflex is present, partial, none. Aqueous commercial ophthalmic eye drops often lead to intense burning, stinging sensation along with tearing. Under comments this is indicated by burn. Dapiprazole HCl use dilates the conjunctiva blood vessels leading to a red conjunctiva and is indicated by red, which can be reversed by Visine drops. Time in minutes is Time (min), aqueous drug solutions content are indicated by w/v %, and the vehicle(s) is given as v/v %. Trade marks, copy writes, and USP designations are not used in the table below. Throughout the study a 40 UL and 17 UL dropper were used to administer ophthalmic drugs. The 40 UL dropper is considered to be the standard size dropper for aqueous vehicle eye drops that are available. The 17 UL dropper was used as indicated in the results below (see comments).

| Results | | | | | Comments |
|---|---|---|---|---|---|
| Time (min) | 0 | 10 | 30 | 60 | comments |

Cyclopentolate HCl, 0.1% mineral oil 1 drop suspension 17 UL

| | | | | | |
|---|---|---|---|---|---|
| Pupil dia (mm) | 3.3 | 4 | 5 | 6 | no burn, long lasting, |
| | | | | | slow start |

Cyclopentolate HCl, 1%, tropicamide 2.5%, commercial aqueous,
1 drop solution

| | | | | | |
|---|---|---|---|---|---|
| Pupil dia (mm) | 3.3 | 4 | 7.8 | 8 | burn, long lasting |

| Time (min) | | | |
|---|---|---|---|
| 0 | 10 | 30 | 60 |

| | | |
|---|---|---|
| Concentration % | Pupil dia (mm) | comments |

Hydroxyamphetamine HBr, mineral oil 1 drop suspension

| | | | | | |
|---|---|---|---|---|---|
| 0.001 | 3.3 | 4 | 4 | 4 | no burn, inactive |
| 0.1 | 3.3 | 5 | 6 | 7.5 | no burn 17 UL |

Phenylephrine HCl mineral oil 1 drop suspension

| | | | | | |
|---|---|---|---|---|---|
| 0.2 | 3.3 | 4 | 5 | 5 | no burn |
| 0.5 | 3.3 | 4 | 5 | 6 | no burn 17 UL |
| 1.0 | 3.3 | 9 | 9 | — | no burn |

Tropicamide mineral oil 69%, petrolatum 30%, castor oil 1%
1 drop suspension

| | | | | | |
|---|---|---|---|---|---|
| 0.1 | 3.3 | 5 | 6 | 7 | no burn |
| 0.2 | 3.3 | 4 | 6 | 8 | no burn 17 UL |
| 1.0 | 3.3 | 6 | 8 | 9 | no burn |

Tropicamide mineral oil 80% petrolatum 20% 1 drop suspension

| | | | | | |
|---|---|---|---|---|---|
| 0.1 | 3.3 | 4 | 6 | 7 | no burn 17 UL |
| 0.2 | 3.3 | 5 | 6 | 7 | no burn 17 UL |
| 0.5 | 3.3 | 4 | 6 | 8 | no burn |
| 1.0 | 3.3 | 6 | 9 | — | no burn |

| | | | | | |
|---|---|---|---|---|---|
| Time (min) | 0 | 10 | 30 | 60 | comments |

Tropicamide 0.5% commercial aqueous 1 drop solution

| | | | | | |
|---|---|---|---|---|---|
| Pupil dia (mm) | 3.3 | 6 | 8 | 9 | burn |

Paremyd commercial aqueous 1 drop solution

| | | | | | |
|---|---|---|---|---|---|
| Pupil dia (mm) | 3.0 | 7 | 8 | 8.5 | some burn |

Tetracaine HCl 0.5% commercial aqueous 1 drop solution

| | | | | | |
|---|---|---|---|---|---|
| Corneal reflex | present | less | gone | — | burn |

Tetracaine HCl 0.1% mineral oil drop suspension

| | | | | |
|---|---|---|---|---|
| Corneal reflex | present | less | gone | no burn |

Sulfacetamide Na 10% commercial aqueous 1 drop solution

| | | |
|---|---|---|
| Reaction | burn | burn |

Sulfacetamide Na 10% mineral oil 1 drop suspension

| | | |
|---|---|---|
| Reaction | gritty | no burn |

Erythromycin 0.5% mineral oil 1 drop suspension

| | | |
|---|---|---|
| Reaction | none | no burn |

Gentamycin 0.3% SO4 mineral oil 1 drop suspension

| | | |
|---|---|---|
| Reaction | none | no burn |

Reversal

Cyclopentolate HCl 0.1% mineral oil 80% petrolatum 20%
1 drop suspension

Time (min)

| | 0 | 10 | 30 | 60 | comments |
|---|---|---|---|---|---|
| Pupil dia (mm) | 3 | 4.5 | 5.5 | 7 | no burn |
| Accomm (cm) | 20 | 46 | 91 | 91+ | |

Followed by:

| | | | | | |
|---|---|---|---|---|---|
| Time (min) | 0 | 10 | 30 | 60 | comments |

Pilocarpine HCl 0.5% mineral oil 1 drop suspension

| | | | | | |
|---|---|---|---|---|---|
| Pupil dia (mm) | 9 | 8.5 | 7 | 6 | no burn |
| Accomm (cm) | 91+ | 71 | 42 | 20 | |

Paremyd commercial aqueous 1 drop solution

| | | | | | |
|---|---|---|---|---|---|
| Pupil dia (mm) | 3 | 7.5 | 8 | 8.5 | some burn |
| Accomm (cm) | 28 | 46 | 85 | 91+ | |

Followed by:

| | | | | | |
|---|---|---|---|---|---|
| Time (min) | 0 | 10 | 30 | 60 | comments |

Pilocarpine HCl 0.5% commercial aqueous 1 drop solution

| | | | | | |
|---|---|---|---|---|---|
| Pupil dia (mm) | 8 | 7.5 | 7 | 7 | burn |
| Accomm (cm) | 91+ | 80 | 75 | 46 | |

Tropicamide 0.1% 80% mineral oil 20% petrolatum
1 drop suspension 17 UL

| | | | | | |
|---|---|---|---|---|---|
| Pupil dia (mm) | 3 | 5.8 | 6 | 6.5 | no burn |
| Accomm (cm) | 30 | 46 | 61 | 91+ | |

Followed by:

| | | | | | |
|---|---|---|---|---|---|
| Time (min) | 0 | 10 | 30 | 60 | comments |

Dapiprazole HCl 0.005% mineral oil 100% 1 drop suspension 17 UL

| | | | | | |
|---|---|---|---|---|---|
| Pupil dia (mm) | 6.5 | 5.5 | 5 | 4.8 | no burn |
| Accomm (cm) | 91+ | 51 | 30 | 30 | red eye |

Tropicamide 0.5% mineral oil 80% petrolatum 20% 1 drop suspension

| | | | | | |
|---|---|---|---|---|---|
| Pupil dia (mm) | 3 | 5 | 8 | 8.8 | no burn |
| Accomm (cm) | 30 | 46 | 61 | 61+ | |

Followed by:

| | | | | | |
|---|---|---|---|---|---|
| Time (min) | 0 | 10 | 30 | 60 | comments |

Dapiprazole HCl 0.5% commercial aqueous 1 drop solution

| | | | | | |
|---|---|---|---|---|---|
| Pupil dia (mm) | 9 | 8 | 6 | 3 | burn |
| Accomm (cm) | 61+ | 46 | 40 | 30 | red eye |

Phenylephrine HCl 2.5% Tropicamide 1% aqueous
1 drop solution 17 UL

| | | | | | |
|---|---|---|---|---|---|
| Pupil dia (mm) | 4 | 6 | 8 | 8.5 | burn |
| Accomm (cm) | 30 | 60 | 91+ | | |

Followed by:

| | | | | | |
|---|---|---|---|---|---|
| Time (min) | 0 | 10 | 30 | 60 | comments |

Dapiprazole HCl 0.5% aqueous 1 drop solution

| | | | | | |
|---|---|---|---|---|---|
| Pupil dia (mm) | 8.5 | 8 | 7 | 5 | burn |
| Accomm (cm) | 91+ | 51 | 46 | | red eye |

Paremyd commercial aqueous 1 drop solution

| | | | | | |
|---|---|---|---|---|---|
| Pupil dia (mm) | 3 | 6 | 8 | 8.5 | burn |
| Accomm (cm) | 30 | 46 | 61+ | | |

Followed by:

| | | | | | |
|---|---|---|---|---|---|
| Time (min) | 0 | 10 | 30 | 60 | comments |

Dapiprazole HCl 0.5% commercial aqueous 1 drop solution

| | | | | | |
|---|---|---|---|---|---|
| Pupil dia (mm) | 9 | 7 | 7 | 5 | burn |
| Accomm (cm) | 61+ | 61 | 50 | 30 | red eye |

| | |
|---|---|
| 2 drops Visine | clear eye |

Tropicamide 0.25% corn oil 1 drop suspension

| | | | | | |
|---|---|---|---|---|---|
| Pupil dia (mm) | 3.3 | 5 | 7 | 8 | no burn |
| Accomm (cm) | 30 | 55 | 65 | 90+ | |

Followed by:

| | | | | | |
|---|---|---|---|---|---|
| Time (min) | 0 | 10 | 30 | 60 | comments |

Dapiprazole HCl 0.25% mineral oil 80% petrolatum 20%
1 drop suspension

| | | | | | |
|---|---|---|---|---|---|
| Pupil dia (cm) | 8 | 6 | 5 | 4 | no burn |
| Accomm (cm) | 90+ | 90 | 36 | 30 | red eye |

| | |
|---|---|
| 2 drops Visine | clear eye |

Tropicamide 0.25% vegetable oil 1 drop suspension

| | | | | | |
|---|---|---|---|---|---|
| Pupil dia (mm) | 3.3 | 6 | 7 | 8 | no burn |
| Accomm (cm) | 30 | 60 | 70 | 90+ | |

Followed by:

| | | | | | |
|---|---|---|---|---|---|
| Time (mm) | 0 | 10 | 30 | 60 | comments |

Dapiprazole HCl 0.25% mineral oil 80% petrolatum 20%
1 drop suspension

| | | | | | |
|---|---|---|---|---|---|
| Pupil dia (mm) | 8 | 6 | 4 | 4 | no burn |
| Accomm (cm) | 90+ | 57 | 40 | 35 | red eye |

| | |
|---|---|
| 2 drops Visine | clear eye |

Tropicamide 0.25% mineral oil 80% petrolatum 20%
1 drop suspension 17 UL

| | | | | | |
|---|---|---|---|---|---|
| Pupil dia (mm) | 3.5 | 6 | 8 | 8.5 | no burn |
| Accomm (cm) | 34 | 50 | 84 | 90+ | |

Followed by:

Dapiprazole HCl 0.05% mineral oil 80% petrolatum 20%
1 drop suspension

| | Time (min) | | | | |
|---|---|---|---|---|---|
| | 0 | 10 | 30 | 60 | comments |
| Pupil dia (mm) | 8.5 | 5.5 | 4 | 3.5 | no burn |
| Accomm (cm) | 90+ | 50 | 44 | 36 | red eye |

| | |
|---|---|
| 2 drops Visine | clear eye |

### Combinations

Combinations of drugs in a non-aqueous oil vehicle are not available on the market. Aqueous solution of drugs can interact but not when the drugs are suspended in a non-aqueous oil vehicle. With suspended drugs there is no medium for the drugs to interact and as a result many combinations are possible. An interesting combination is dapiprazole HCl and tetrahydrozoline HCl. A side effect of dapiprazole HCl is dilation of blood vessels of the conjunctiva (red eye). In certain concentrations tetrahydrozoline constricts blood vessels and produces mydriasis. Several combinations of the two drugs were tried in aqueous solution and non-aqueous oil preparations. An aqueous solution of 0.5% dapiprazole HCl and 0.1% tetrahydrozoline HCl w/v was prepared in an isotonic saline solution. This combination produced miosis with stinging and burning that was followed by a red eye within 10 minutes after administration. The combination of 0.05% dapiprazole HCl and 0.1% tetrahydrozoline HCl w/v in an oil vehicle produced miosis and there was no red eye, stingy or burning that resulted after administration. Next, a potent concentration of tropicamide 0.5% w/v suspended in an 80% mineral oil and 20% white petrolatum 20% v/v vehicle was administered as one drop and achieved pupil dilation from an average 3.3 mm to 8.5 mm with a beginning accommodation of 30 cm to 61+cm within thirty minutes after administration. A single drop of dapiprazole HCl 0.05% and tetrahydrozoline HCl 0.1% w/v suspended in 80% mineral oil and 20% petrolatum v/v was administered. In one hour the accommodation was back to normal and the pupil diameter averaged 5.5 mm. There was no stinging or burning reported and red eye did not develop. However, in one of the subjects the eye became slightly pink for several minutes.

Cyclopentolate HCl as a powder has a warning label to keep the powder in the cold since it is not stable at room temperature. Cyclogel, an aqueous preparation, is stable for several years. Cyclopentolate HCl was studied several times using freshly prepared suspensions in oil.

Ophthalmic drugs suspended in an oil vehicle elicited no adverse effects such as the sensation of burning, stinging and the copious tearing experienced using the commercial aqueous eye drops. Many times diluted ophthalmic drugs suspended in oil were just as effective on the human eye as the corresponding commercial aqueous eye drops. It is difficult to demonstrate the potency of drugs that produce miosis on the untreated eye. As a result drugs were first used to produce mydriasis and then pilocarpine HCl or dapiprazole HCl were used. In an experiment using eight subjects the accommodation was measured when the subjects were kept in a poorly lighted room. This resulted in a pupil diameter of 5 mm. Then the subject went into the bright sunlight to yield a pupil diameter of around 2 mm. The accommodation measurement did not change. When dapiprazole HCl was used to reduce the mydriasis-cycloplegia effects of tropicamide the subjects began to notice the return of accommodation within minutes, long before the pupil size returned to the initial drug treatment value.

### Safety and Comfort

The classical ointments are composed of white petrolatum, USP with as much as 20% mineral oil, USP added. Drugs suspended in these ointments are stable and effective. However, these ointments are applied as a stiff ribbon to the eye which blurs vision for hours. In the last few decades thick gels and the like have been called ointments and are used to suspend ophthalmic drugs. These gels have an aqueous component and like the classical ointment are thick and blur vision. Further, many of these new ointments are too thick to be used as eye drops.

Mineral oil USP with and without white petrolatum USP (0% to 30%) can be used as eye drops to which suspended ophthalmic drugs are added. Our study showed that mineral oil USP with or without 20% white petrolatum USP eye drops blur the vision for about 20 seconds while isotonic saline eye drops reduce the visual acuity for three times longer. Also, the ophthalmic drugs when suspended in an oil vehicle are bland. A drop of ophthalmic drugs suspended in mineral is not detected by the subject while a drop of isotonic saline is always noted. The usual question when using oil based ophthalmic medicaments: “Is the drop in yet?”

It is generally accepted that the use of phenylephrine HCl 10% aqueous eye drops can lead to hypertension, tachycardia and possible heart attack. Drugs delivered as eye drops reach the nasal cavity (Scruggs, et al. and Mirshihi, et al.). The greatly diluted drugs in eye drops using an oil vehicle reduces this toxic potential. Infants have a poorly developed tear drainage system. Eye drops given to infants readily reach the nasal cavity. This potential problem is lessened with the use of the low concentration of ophthalmic drugs used in the oil vehicle as compared to commercial aqueous eye drops. Eye droppers deliver about the same volume of water or oil, and do not depend only on the dropper opening.

The use of an oil vehicle to suspend ophthalmic drugs in eye drops is comfortable and has a reduced potential for toxicity as compared to commercial aqueous eye drops.

### Stability

Phenylephrine HCl, USP 0.2% w/v was suspended in mineral oil, USP 80% and white petrolatum, USP 20% v/v eye and was tested as eye drops. It was tested again 18 months later and gave the same results (mydriasis, cycloplegia and constricted conjunctiva blood vessels). The suspension was kept in a room that was neither heated in the winter nor cooled in the summer (ambient temperatures) Tropicamide USP, 0.1% and 0.5% w/v suspended in mineral oil, USP 80% and white petrolatum, USP 20% were stored in the same room at ambient temperature for 16 months and yielded the same results (mydriasis). Rev-eyes came in two vials. One vial contained five milliliters of water and the other vial dapiprazole HCl, buffer, and cellulosic derivative which on mixing with the first vial gave 0.5% dapiprazole HCl solution. Added to the second vial was mineral oil USP 80% and white petrolatum USP 20% v/v in the amount of 5 mL, with stirring. This suspension on testing then and 16 months later yielded miosis and dilated conjunctiva blood vessels (red eye).

Commercial phenylephrine HCl, USP, cyclopentolate HCl, USP, aqueous eye drops are to be kept at 15 degrees C. to 30 degrees C. The same for Rev-Eyes except once water is added to the powdered dapiprazole HCl the solution is to be used within 21 days. In the absence of water the suspended ophthalmic drugs in oil vehicle are stable for long periods of time even when kept at ambient temperatures.

### Visual Acuity

It is reported that oil vehicles cannot be used as ophthalmic eye drops because of long term blurring. The viscous ointments do lead to long term blurring. However, these reports cannot be extrapolated to eye drops using mineral oil, USP.

Blurring from a single drop of mineral oil USP with and without white petrolatum, USP 20% lasted an average of 19 seconds and 21 seconds; respectively. Twenty subjects received a drop of mineral oil in one eye and a solution of mineral oil-white petrolatum in the other eye later. The oil drops weighed an average of 25 mg while a drop of isotonic saline weighs 39 mg. The twenty subjects ranged from 18 to 85 years of age which included 10 females. The most often used eye dropper is the 40 UL. This size dropper delivers around 40 mg of aqueous solution. The “mini” dropper 17 UL size yields around 17 mg of water. The 40 UL dropper yields around 25 mg of oil while the 17 UL dropper, when held perpendicular, yields 12 mg of oil. Commercial aqueous dapiprazole HCl 0.5% is used with the 40 UL eye droppers, with a recommended dosage of two drops initially followed 5 minutes later by two drops, to return accommodation and lessen mydriasis of ophthalmic drugs used in eye examination. Using the 17 UL dropper and dapiprazole HCl 0.05% suspended in a mineral oil 80% and petrolatum 20% vehicle was as effective as four drops of the commercial aqueous preparation. The commercial dapiprazole HCl 0.5% eye drops delivers 160 mg of aqueous solution at the recommended dosage, while a drop of dapiprazole 0.05% suspended in mineral oil 80% and petrolatum 20% using the 17 UL dropper yields 12 mg of oil. Since the 17 UL drug preparation contains ten times less of dapiprazole HCl that could reach the systemic circulation via the punctum and nasal cavity. This is at least 130 times less dapiprazole. The possible toxic effect of this and other drugs suspended in oil has a greatly reduced potential for toxicity as compared to the corresponding commercial ophthalmic eye drops that are currently available.

### Compounding

The two component, oil delivery system and suspended powdered ophthalmic medicament, do not require other agents (excipients, preservatives) and it is only necessary for the pharmacists or manufacturers to mix the sterile ophthalmic powder with sterile oil. Sterile ophthalmic suspension was prepared by pharmacists. A loss of potency occurs when the oil-drug suspension is homogenized or heated to sterilize at 135 degrees C. for 3 hours. The vehicle-drug are delivered using an eye dropper. A single drop is adequate in Caucasians but it may take more drops in other races. Not all vegetable oils can be used. Olive oil becomes rancid in time. Both olive oil and sesame seed oil produce a period of burning and stinging when applied to the eye in an amount of 7 mg. Sesame seed and olive oil have persistent strong odors and were studied no further.

A product that contains dapiprazole hydrochloride USP and tetrahydrozoline hydrochloride USP which uses the unique non-aqueous oil delivery system is currently in commerce. The product is used to reverse the effects of mydriasis and cycloplegia while minimizing the redness that is associated with the use dapiprazole hydrochloride. The formulation contains 0.05% dapiprazole hydrochloride and 0.1% tetrahydrozoline hydrochloride w/v that is delivered by the oil delivery system of an 80% mineral oil and 20% petrolatum USP v/v. The product is packaged using a 10 ml dropper bottle with a 5 ml fill.

1. A nonaqueous oil delivery system for suspending ophthalmic drugs for use in ophthalmic preparations.

2. The nonaqueous oil delivery system in claim 1 yields a short duration of visual acuity loss as compared to commercial aqueous eye drops.

3. The nonaqueous oil delivery system in claim 1 contains drugs suspended in castor oil, corn oil, glycerol, mineral oil USP, vegetable oil, and white petrolatum USP, and mixtures thereof.

4. The nonaqueous oil delivery system in claim 1 includes antimicrobials, miotics, mydriatics, mydriatic-cycloplegics, mydriatic-cycloplegic reversing agents, topical anesthetics and combinations thereof.

5. The nonaqueous oil delivery systems in claim 1 are administered as suspended drugs and are therapeutically effective when used as one drop. Using this nonaqueous oil delivery system a single drop of ophthalmic drug with a concentration that is 10 times less than the same drug used in commercially available aqueous eye drops is as effective as the commercially available aqueous ophthalmic eye drops that require many applications to be effective.

6. The nonaqueous oil delivery system in claim 1 lowers the potential for systemic toxicity.

7. The nonaqueous oil delivery system in claim 1 are cyclopentolate HCl, dapiprazole HCl, erythromycin, gentamycin sulfate, hydroxyamphetamine HBr, phenylephrine HCl, pilocarpine HCl, sulfacetamide sodium, tetracaine HCl, tetrahydrozoline HCl and tropicamide.

8. The nonaqueous oil delivery system in claim 1 leads to long term shelf-life of the suspended drugs.

9. The nonaqueous oil delivery systems in claim 1 are of high potency and are comfortable to use.

10. Methods are given for the preparation of the sterile, non-aqueous, suspended drugs in oil and the dispensing said vehicles to the external eye.
